(19) United States
(12) Patent Application Publication
(10) Pub. No.: US 2019/0240187 A1
(43) Pub. Date: Aug. 8, 2019

(54) ORAL PREPARATIONS WITH OMEPRAZOLE OR PANTOPRAZOLE

(71) Applicant: SYMRISE AG, Holzminden (DE)

(72) Inventors: Joachim HANS, Holzminden (DE); Jakob LEY, Holzminden (DE); Susanne PAETZ, Höcter (DE)

(73) Assignee: Symrise AG, Holzminden (DE)

(21) Appl. No.: 15/779,416

(22) PCT Filed: Nov. 27, 2015
(86) PCT No.: PCT/EP2015/077991

§ 371 Date: Sep. 6, 2018

(51) Int. Cl. A61K 31/352; A61K 31/4439; A61K 9/68; A61K 9/46; A61K 9/20; A61P 1/04

(52) U.S. Cl. CPC A61K 31/352; A61K 31/4439; A61K 9/0058; A61K 9/08; A61K 9/2009; A61P 1/04; A61K 9/0007

(57) ABSTRACT

Proposed is a preparation comprising
(a) omeprazole and/or pantoprazole; and
(b) eriodyctiol and/or homoeriodyctiol.

### FIELD OF THE INVENTION

The invention is in the field of active pharmaceutical ingredients and concerns oral preparations containing omeprazole or pantoprazole with improved galenics.

### STATE OF THE ART

Omeprazole and pantoprazole are drugs from the group of proton pump inhibitors used for the treatment of gastric and duodenal ulcers and reflux esophagitis. The most important indications are:
Treatment of duodenal ulcer (Ulcus duodeni);
Treatment of stomach ulcer (Ulcus ventriculi);
Treatment of inflammation of the gastric tube caused by gastric juice reflux (reflux esophagitis);
Treatment of symptoms caused by the reflux of gastric acid into the esophagus (reflux disease, heartburn);
Treatment of Zollinger-Ellison syndrome;
Prevention of recurrence of inflammation of the esophagus or ulcers of the stomach and duodenum caused by the use of certain painkillers or rheumatism medicines (so-called non-steroidal anti-inflammatory drugs), and
Combination therapy of Helicobacter pylori infections.

Omeprazole and Pantoprazole are usually administered as tablets or capsules, rarely also by infusion solution.

A disadvantage is that omeprazole and pantoprazole have an intense bitter taste that could not be neutralized, masked or inhibited until now. The taste deficits make the oral intake unpleasant, which leads to the fact that patients often suspend the regular intake of the medication.

The objective of the present invention was therefore to provide preparations comprising omeprazole or pantoprazole which do not show the disadvantages described and which are particularly characterized by a neutral taste. Another objective was to improve the bioavailability of omeprazole and pantoprazole by improving resistance to gastric acid.

### DESCRIPTION OF THE INVENTION

A first object of the invention concerns preparations containing

(a) omeprazole and/or pantoprazole; and
(b) eriodyctiol and/or homoeriodyctiol.

Surprisingly, it was found that eriodyctiol and/or homoeriodyctiol is/are excellently suited to mask the bitter taste of omeprazole or pantoprazole. In particular when used together bitterness is reduced by about half. It was also found that mixtures of components (a) and (b) have a higher resistance to gastric acid than omeprazole or pantoprazole alone. By combining omeprazole or pantoprazole with one or both additives, their bioavailability can also be improved.

Omeprazole

The artificial word omeprazole is used to denote the substance (RS)-5-methoxy-2-[(4-methoxy-3,5-dimethylpyridin-2-yl)methanesulfinyl]-1H-benzoinnidazole (I)

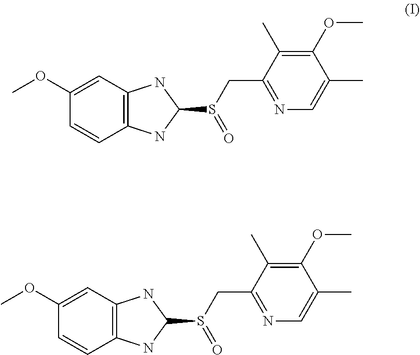

Omeprazole was the first proton pump inhibitor introduced into therapy and was approved as a well-tolerated stomach medicine in the 1990s. Since 1998 Omeprazole has also been offered as a Multiple Unit Pellet System (MUPS). These tablets are comprates of small, enteric coated pellets that disintegrate rapidly when in contact with liquid. The (S)-enantiomer of omeprazole (esomeprazole, Nexium®) has been marketed since 2000. Since esomeprazole is increasingly metabolized via CYP2A19 and thus more slowly than racemic omeprazole, the result is a retarded reduction in plasma levels and thus better bioavailability compared to the racemate. The latest innovations are omeprazole sodium bicarbonate for-formulations (Zege-rid®) from the US company Santarus. They do not require a gastric juice-resistant coating and enable additional dosage forms that are also intended to release the active ingredient particularly rapid. An oral suspension has been on the market in the USA since 2004, capsules were approved in 2006 and a chewable tablet is expected to be approved shortly. As far as the invention refers to omeprazole, all isomers and galenic forms listed above are included.

Pantoprazole ((RS)-5-(difluoromethoxy)-2-[(3,4-dimethoxy-2-pyridyl) methylsulfinyl] benzimidazole) (II) is also a proton pump inhibitor and is a structurally closely related derivative of omeprazole. It has the same indications as omeprazole and is administered in very similar or identical dosage forms.

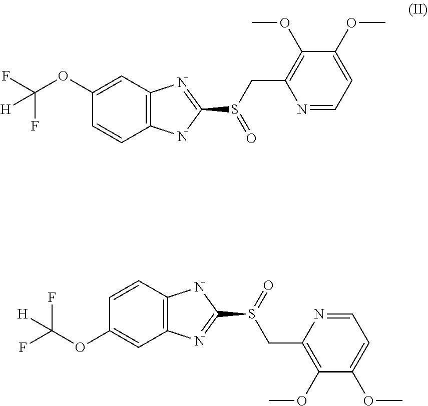

Since omeprazole and pantoprazole are only stable in alkaline to neutral environments, the active ingredients are often used in tablets or capsules that are coated with an acid-resistant coating so that they dissolve only in the small intestine and not in the acidic environment of the large intestine. Alternatively, the substances can also be applied to basic carriers, which produce an alkaline micromilieu when absorbed orally.

Eriodyctiol Und Homoeriodyctiol

Eriodyctiol, 2-(3,4-dihydroxyphenyl)-5,7-dihydroxy-4-chronnanone), and homoeriod-yctiol, 5-7-dihydroxy-2-(4-hydroxy-3-methoxyphenyl)-4-chronnanone, which are shown in the following figure,

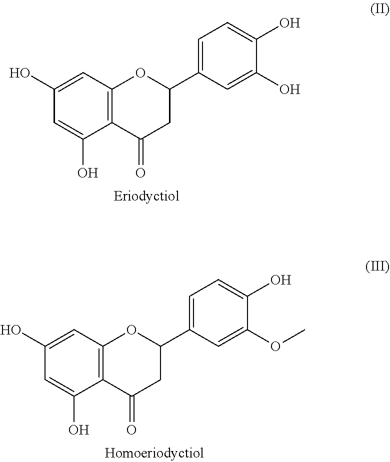 [SMILES: O=C1CC(c2ccc(O)c(O)c2)Oc2cc(O)cc(O)c21 ; COc1cc(C2CC(=O)c3c(O)cc(O)cc3O2)ccc1O]

Belong to the flavanones and can be found as active ingredients in Yerba Santa, Eriodictyon ssp. According to the invention, eriodictyol and homoeriodictyol can be used on their own or in mixtures, wherein the flavanones can be used as an enantiomer mixture from 0.1 2S:100 2R to 0.1 2R:100 2R, as pure enantiomers, or preferably as racemic (50:50) or almost racemic mixture from 35 2S: 65 2R to 65 2R: 35 2S, more preferably 45 2S: 55 2R to 55 2R: 45 2S. Each of the two flavanones may be present in the form of a salt in whole or in part, preferably monovalent or divalent salts with sodium, potassium, calcium, magnesium and ammonium. Particularly preferred are homoeriodictyol in an enantiomer ratio 45 2S: 55 2R to 55 2R: 45 2S and/or homoeriodictyol monosodium salt in an enantiomer ratio 45 2S: 55 2R to 55 2R: 45 2S and/or eriodictyol in an enantiomer ratio 45 2S: 55 2R to 55 2R: 45 2S.

Oral Preparations

The oral preparations according to the invention contain components (a) and (b) preferably in a weight ratio of about 4:1 to 400:1 and in particular about 8:1 to 200:1. Component (a) omeprazole or pantoprazole is preferably contained in an amount of 1,000 to about 30,000 ppm, in particular about 2,000 to 20,000 ppm. Furthermore, it is preferred that the oral preparations contain component (b) eriodyctiol and/or homoeriodyctiol in an amount of about 50 to about 500 ppm and in particular about 100 to about 200 ppm. With regard to bitter masking, it has proven to be particularly advantageous to use eriodyctiol and homoeriodyctiol together, in particular in an amount of about 25 to about 250 ppm and preferably about 50 to about 150 ppm.

As described above, the oral dosage forms of omeprazole or pantoprazole may be liquid. However, the preferred galenic forms are oral dosage forms, in particular oral solid or liquid dosage forms, and include, for example, solutions, suspensions, emulsions, powder for swallowing, chewable tablets, compressed tablets, capsules, lacquered tablets, lozenges, chewable sweets, fruit gums or medical chewing gums. If the forms are solid, they preferably have an acid-resistant coating (for tablets) or shell (for capsules) as explained above.

Tablets

The oral preparations according to the invention also include tablets. Tablets may contain further ingredients that are common for this dosage form, for example: Carriers, disintegrants and/or dyes and flavors. Tableting itself is a well-known industrial process. An example is DE 10 2006 051529 A1 (HENKEL), in which the production of tablets is described in detail.

Carriers

Suitable carriers can be divided into fillers and binders. Fillers ensure that the tablet gets the necessary size/mass. Starches (corn, potato and wheat starch) and lactose are used. Other fillers are: Glucose, mannitol, sorbitol. Fructose is rarely used due to its high price. Sucrose is mainly used for lozenges.

Binders, on the other hand, ensure the cohesion in the granules or powders and, in addition to the pressing pressure, the strength of tablets. They can be divided into dry binders such as MCC (microcrystalline cellulose) or starch and wet binders/adhesives for granulation such as starch paste, cellulose ether, collidone and gelatin.

Disintegrants

In order to facilitate the disintegration of prefabricated shaped bodies, it is possible to incorporate disintegration aids, so-called tablet disintegrants, in order to shorten the disintegration times. Tablet disintegrants respectively disintegration accelerators are understood to be auxiliary substances that ensure rapid disintegration of tablets in water or other media and rapid release of the active ingredients.

These substances, which are referred to as disintegrants due to their effect, increase their volume when water enters, whereby on the one hand the intrinsic volume increases (swelling), and on the other hand a pressure can be generated through the release of gases, which causes the tablet to disintegrate into smaller particles. Well-known disintegration aids are, for example, carbonate/citric acid systems, whereby other organic acids can also be used. Swelling disintegration aids include for example synthetic polymers such as polyvinylpyrrolidone (PVP) or natural polymers or modified natural substances such as cellulose and starch and their derivatives, alginates or casein derivatives.

Cellulose-based disintegrants are used as preferred disintegratants. Pure cellulose has the formal gross formula (C₆H₁₀O₅)ₙ and formally represents a beta-1,4-polyacetate of cellobiose, which in turn is composed of two molecules of glucose. Suitable celluloses consist of approx. 500 to 5000 glucose units and therefore have average molecular weights of 50,000 to 500,000. Cellulose derivatives, which are obtainable by polymer analogous reactions from cellulose, can also be used as cellulose-based disintegration agents within the scope of the present invention. Such chemically modified celluloses include, for example, products from esterification respectively etherification in which hydroxy groups have been substituted by hydrogen atoms. However, celluloses in which the hydroxy groups have been replaced by functional groups that are not bound via an oxygen atom can also be used as cellulose derivatives. The group of cellulose derivatives include, for example, alkali cellulose, carboxymethyl cellulose (CMC), cellulose esters and ethers and amino celluloses. The cellulose derivatives mentioned are preferably not used alone as cellulose-based disintegrants, but in mixtures with cellulose. The content of cellulose derivatives in these mixtures is preferably less than 50% by weight, more preferably less than 20% by weight, based on the cellulose-based disintegrant. Pure cellulose, which is free of cellulose derivatives, is particularly preferred as a cellulose-based disintegrant.

The cellulose used as disintegrant is preferably not used in a finely divided form, but rather converted into a coarser form, for example granulated or compacted, before mixing into the pre-mixes to be compacted. The particle sizes of such disintegration agents are mostly above 200 μm, preferably the particle sizes of at least 90% by weight is between 300 and 1,600 μm and in particular the particle sizes of at least 90% by weight is between 400 and 1,200 μm.

Microcrystalline cellulose can be used as a further cellulose-based disintegrant or as a constituent of this component. This microcrystalline cellulose is obtained by partial hydrolysis of celluloses under conditions that only attack and completely dissolve the amorphous regions (approx. 30% of the total cellulose mass) of the celluloses but leave the crystalline regions (approx. 70%) undamaged. Subsequent disaggregation of microfine celluloses produced by hydrolysis yields the microcrystalline celluloses, which have primary particle sizes of about 5 μm and can be compacted into granules with an average particle size of 200 μm, for example.

In addition, gas-developing effervescence systems can also be preferably used for the purposes of the invention. The gas-generating effervescence system can consist of a single substance that releases a gas upon contact with water. Among these compounds is magnesium peroxide, which releases oxygen upon contact with water. However, the gas-releasing bubble system usually consists of at least two components that react with each other to form gas. While a large number of systems are conceivable and feasible that release, for example, nitrogen, oxygen or hydrogen, the effervescence system used in detergents and cleaning agents can be selected on the basis of both economic and ecological aspects. Preferred effervescence systems consist of alkali metal carbonate and/or alkali hydrogen carbonate and an acidifier capable of releasing carbon dioxide from the alkali metal salts in an aqueous solution.

Colorants

Food colorants, or colorants for short, are food additives for coloring preparations that are suitable for consumption. Colorants are divided into the groups of natural colorants and synthetic colorants. The nature-identical colorants are also of synthetic origin. The nature-identical colorants are synthetic replicas of naturally occurring coloring substances. Suitable colorants for use in the present composition are selected from: Curcumin, E 100 riboflavin, lactoflavin, vitamin B2, E 101 tartrazine, E 102 quinoline yellow, E 104 yellow orange S, yellow orange RGL, E 110 cochenille, carminic acid, real carmine, E 120 azorubin, carmoisin, E 122 amaranth, E 123 Cochineal Red A, Ponceau 4 R, Victoria scarlet 4 R, E 124 erythrosine, E 127 allur-red AC, E 129 Patent blue V, E 131 Indigotine, Indigo-Carmine, E 132 Brilliant blue FCF, Patent blue AE, Amido blue AE, E 133 Chlorophylls, Chlorophylline, E 140 Copper complexes of chlorophylls, Copper-chlorophylline complex, E 141 Brillic acid green, green S, E 142 Sugar cube, sugar cube, E 150 a Sulphite liquor-sugar cube, E 150 b Ammonia-sugar cube, E 150 c ammonium sulphite sugar cullure, E 150 d brilliant black FCF, brilliant black PN, black PN, E 151 vegetable coal, E 153 brown FK, E 154 brown HT, E 155 carotene, E 160 a annatto, bixin, norbixin, E 160 b capsanthin, capsorubin, E 160 c lycopene, E 160 d beta-apo-8′-carotene, apocarotene, beta-apocarotene, E 160 e beta-apo-8′-carotenic acid ethyl ester (C30), apocarotene ester, beta-carotenic acid ester, E 160 f lutein, xanthophyll, E 161 b canthaxanthin, E 161 g betanin, praying red, E 162 anthocyanins, E 163 calcium carbonate, E 170 titanium dioxide, E 171 iron oxides, iron hydroxides, E 172 aluminum, E 173 silver, E 174 gold, E 175 litholrubin BK, ruby pigment BK, E 180.

Flavoring Substances

The preparations of the invention may contain one or more flavoring substances; flavoring substances are defined, for example, by European or American legislation, e.g. Regulation (EC) No 2232/96 of the European Parliament and of the Council and the positive list defined in the Implementing Regulation (EU) No 872/2012 of the Commission. Typical examples include: acetophenone, allyl capronate, alpha-ionone, beta-ionone, anisaldehyde, anisyl acetate, anisyl formate, benzaldehyde, benzothiazole, benzyl acetate, benzyl alcohol, benzyl benzoate, beta-ionone, butyl butyrate, butyl capronate, butylidenephthalide, carvone, camphene, caryophyllene, cineol, cinnamyl acetate, citral, citronellol, citronellyl acetate, cyclohexyl acetate, cymol, damascone, decalactone, dihydrocoumarin, dimethylanth ranilate, dimethylanthranilate, dodecalactone, ethoxyethyl acetate, ethyl butyric acid, ethyl butyrate, ethyl caprinate, ethyl capronate, ethyl crotonate, ethyl furaneol, ethyl guaiacol, ethyl isobutyrate, ethyl isovalerianate, ethyl lactate, ethylmethylbutyrate, ethylpropionate, eucalyptol, eugenol, ethylheptylate, 4-(p-hydroxyphenyl)-2-butanone, gamma-decalactone, geraniol, geranylacetate, geranylacetate, grapefruitaldehyde, methyldihydro-jasmonate (e.g. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenylacetate, cis-3-hexenylacetate, cis-3-hexenylcapronate, trans-2-hexenyl capronate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isamyl isovalerianate, isobutyl butyrate, isobutyraldehyde, Isoeugenol ether, isopropylmethylthiazole, lauric acid, leavulinic acid, linalool, lina-lool oxide, linalyl acetate, menthol, menthofuran, methylanthranilate, methylbutanol, methylbutyric acid, 2-methylbutyl acetate, methyl capronate, methyl cinnamate, 5-methylfurfural, 3,2,2-methylcyclopentenolone, 6,5,2-methylheptenone, methyl dihydrojasmonate, methyl jasmonate, 2-methylmethylbutyrate, 2-methyl-2-pentenolic acid, methyl thiobutyrate, 3,1-methylthiohexanol, 3-methylthiohexyl acetate, nerol, nerylacetate, trans,trans-2,4-Nonadienal, 2,4-Nonadienol, 2,6-Nonadienol, 2,4-Nonadienol, nootkaton, delta octalacton, gamma octalacton, 2-octanol, 3-octanol, 1,3-octenol, 1-octylacetat, 3-octylacetat, palmitic acid, paraldehyde, phellandrene, pentanedione, phenylethyl acetate, phenylethyl alcohol, phenylethyl alcohol, phenylethylisovalerianate, piperonal, propionaldehyde, propyl butyrate, pulegon, pulegol, sinensal, sulfurol, terpinene, terpineol, terpinolene, 8,3-thiomenthanone, 4,4,2-thiomethylpentanone, thymol, delta-undecalactone, gamma-undecalactone, valencene, valeric acid, vanillin, acetoin, ethylvanillin, ethyl vanillin isobutyrate (=3-ethoxy-4-isobutyryloxybenzaldehyde), 2,5-dimethyl-4-hydroxy-3(2H)-furanone and its derivatives (preferably honno-furaneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (preferably ethylmaltol), coumarin and coumarin derivatives, gamma-lactones (preferably gamma-undecalactone, gamma-nonalactone, gamma-decalactone), delta-lactones (preferably 4-methyldelta-decalactone), masso-ilactone, delta-decalactone, tubero-lactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)furanone, acetic acid isoamyl ester, butyric acid ethyl ester, butyric acid n-butyl ester, butyric acid isoamyl ester, 3-methyl butyric acid ethyl ester, n-hexanoic acid ethyl ester, n-hexanoic acid allyl ester, n-hexanoic acid n-butyl ester, n-octanoic acid ethyl ester, ethyl-3-methyl-3-phenylglycidate, ethyl-2-trans-4-cis-decadienoate, 4-(p-hydroxyphenyI)-2-butanone, 1,1-dinnethoxy-2,2,5-trimethyl-4-hexane, 2,6-dimethyl-5-hepten-l-al and phenylacetaldehyde, 2-methyl-3-(methylthio)furan, 2-methyl-3-furanthiol, bis(2-methyl-3-furyl)disulfide, furfuryl mercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, 2-Acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-nnethoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3(2H)-furanone, guaiacol, 3-hydroxy-4,5-dimethyl-2(5H)-furanone, 3-hydroxy-4-methyl-5-ethyl-2(5H)-furanone, cinnamic aldehyde, cinnamic alcohol, methyl salicylate, isopulegol and (not explicitly mentioned here) stereoisomers, enantiomers, positional isomers, diastereomers, cis/trans-isomers or epimers of these substances.

Flavoring substances can also directly influence the taste (taste modulators) and are for example selected from the group consisting of monosodium glutamate, free glutamic acid, nucleotides or their pharmaceutically acceptable salts, strombin, theogalline as described in JP 2007 110988 A, pyridine-betain compounds as described in EP 1291342 B1, glutamic acid glycosides as described in WO 2002/087361 A1, malic acid glycosides as described in WO 2006 003107 A1, glutathione derivatives as described in EP 0181421 or WO 2007/042273 A1), Alkylpyridines (preferably alkylpyridines as described in WO 2009/122318 A1 and WO 2009/1223319 A1), in particular 2-hexyl, 2-heptyl and 2-octylpyridine, (2E,6Z)-N-cyclopropylnona-2,6-dienamide, (2E,6Z)-N-ethylnona-2,6-dienamide, N-[(2E)-3,7-dimethylocta-2,6-dienyl]cyclopro-pancarboxannide, N′-[(2-methoxy-4-methyl-phenyl)methyl]-N-[2-(5-methyl-2-pyridyl)ethyl]oxamide, N′-[(2,4-dimethoxyphenyl)methyl]-N-[2-(2-pyridyl)ethyl]-oxamide, N′-[(2-methoxy-4-methyl-phenyl)methyl]-N-[-2-(2-pyridyl)ethyl]oxannide, N-(1-propylbutyl)-1,3-benzodioxole-5-carb-oxamide, 1-(2-hydroxy-4-isobutoxy-phenyl)-3-(2-pyridyl)propan-1-one and 1-(2-hydroxy-4-methoxy-phenyl)-3-(2-pyridyl)propan-1-one, cinnamic acid amides as described in EP 2529632 B1 or WO 2013/000673), lactisols, hesperitin according to EP 1909599 A1, Phloretin according to EP 1972203 B1 or EP 1998636 B1, hydroxyflavanes according to US 2010/292175 AA, 4-hydroxychalcones according to EP 1972203 B1, extracts based on Hydrangea dulcis according to EP 2298084 A2, or Rubus ssp.; mixtures of whey proteins with lecithins, yeast extracts, plant hydrolysates, powdered vegetables (e.g. onion powder, tomato powder), plant extracts (e.g. of lovage or mushrooms such as shiitake), seaweed and mineral salt mixtures, especially mineral salt mixtures according to US 2009/214728 AA, rubemamine or rubescenamine according to EP 2529632 B1.

Chewing Gums

The oral preparations according to the invention also include chewing gums. These products typically contain a water-insoluble and a water-soluble component.

The water-insoluble base, also known as “gum base”, usually comprises natural or synthetic elastomers, resins, fats and oils, plasticizers, fillers, colorants and optionally waxes. The proportion of the base usually accounts for 5 to 95, preferably 10 to 50 and in particular 20 to 35% by weight of the total composition. In a typical embodiment of the invention, the base is composed of 20 to 60% by weight synthetic elastomers, 0 to 30% by weight natural elastomers, 5 to 55% by weight plasticizers, 4 to 35% by weight fillers and, in minor amounts, additives such as colorants, antioxidants and the like, with the proviso that they are water-soluble only in small amounts

Suitable synthetic elastomers include polyisobutylenes having average molecular weights (according to GPC) of 10,000 to 100,000 and preferably 50,000 to 80,000, isobutyl-ene-isoprene copolymers (“butyl elastomers”), styrene-butadiene copolymers (styrene:butadiene ratio e.g. 1:3 to 3:1), polyvinyl acetates having average molecular weights (according to GPC) of 2,000 to 90,000 and preferably 10,000 to 65,000, polyisoprene, poly-ethylene, vinyl acetate-vinyl laurate copolymers and mixtures thereof. Examples of suitable natural elastomers are rubbers such as smoked or liquid latex or guayule as well as natural rubber materials such as jelutong, lechi caspi, perillo, sorva, massaranduba balata, massaranduba chocolate, nispero, rosindinba, chicle, gutta hang lkang and mixtures thereof. The choice of synthetic and natural elastomers and their mixing ratios depends essentially on whether the chewing gums are to be used to produce bubbles (“bubble gums”) or not. Elastomer mixtures containing jelutong, chicle, sorva and massaranduba are preferably used.

In most cases the elastomers are found to be too hard or too little deformable during processing, so it has turned out to be advantageous to use special plasticizers, which of course also have to meet all requirements for approval as food additives. In this respect, esters of resin acids, such as esters of lower aliphatic alcohols or polyols having wholly or partially cured monomeric or oligomeric resin acids, are particularly suitable. In particular, methyl, glycerol or pentareythritol esters and their mixtures are used for this purpose. Alternatively, terpene resins, which can be derived from alpha-pinene, beta-pinene, delta-limonene or mixtures thereof, can also be used.

Suitable fillers or texturing agents are magnesium or calcium carbonate, ground pumice stone, silicates, especially magnesium or aluminum silicates, clays, aluminum oxides, talc, titanium dioxide, mono-, di- and tricalcium phosphate and cellulose polymers.

Suitable emulsifiers are talc, hardened talc, hardened or partially hardened vegetable oils, cocoa butter, partial glycerides, lecithin, triacetin and saturated or unsaturated fatty acids having 6 to 22 and preferably 12 to 18 carbon atoms as well as their mixtures.

FD and C types approved for coloring food, plant and fruit extracts as well as titanium dioxide can for example be used as colorants and whiteners.

The base materials may contain waxes or be wax-free; examples of wax-free compositions can be found in patent specification U.S. Pat. No. 5,286,500, which content is herewith explicitly referred to. In addition to the water-insoluble gum base, chewing gum preparations regularly contain a water-soluble part which is formed, for example, by plasticizers, sweeteners, fillers, flavorings, flavor enhancers, emulsifiers, dyes, acidifiers, antioxidants and the like, here with the proviso that the components have at least sufficient water solubility. Depending on the water solubility of the particular components, individual components may belong to both the water-insoluble and water-soluble phases. However, it is also possible to use combinations of, for example, a water-soluble and a water-insoluble emulsifier, whereby the individual components are then in different phases. Usually the water-insoluble part makes up 5 to 95 and preferably 20 to 80% by weight of the preparation.

Water-soluble softeners are added to chewing gum preparations to improve chewability and chewing sensation and are typically present in mixtures in amounts of 0.5 to 15% by weight. Typical examples are glycerol, lecithin and aqueous solutions of sorbitol, cured starch hydrolysates or grain syrup.

Sweeteners can be either sugar-containing or sugar-free compounds, which are used in amounts of 5 to 95, preferably 20 to 80 and in particular 30 to 60% by weight based on the chewing gum composition. Typical saccharide sweeteners are sucrose, dextrose, maltose, dextrin, dried invert sugar, fructose, levulose, galactose, corn syrup and their mixtures. Sorbitol, mannitol, xylitol, hardened starch hydrolysates, maltitol and mixtures thereof can be used as sugar substitutes. Furthermore, HIAS (“High Intensity Articifical Sweeteners”), such as sucralose, aspartame, acesulfame salts, alitame, saccharin and saccharin salts, cyclamic acid and its salts, glycyrrhizine, dihydrochalcone, thaumatin, monellin and the like, alone or in blends, may also be used as additives. Particularly effective are also hydrophobic HIAS, which are the subject matter of the international patent application WO 2002 091849 A1 (Wrigleys) as well as stevia extracts and their active ingredients, in particular rebaudioside A. The amount of these substances used depends primarily on their effectiveness and is typically in the range of 0.02 to 8% by weight.

Fillers such as polydextrose, rafftilose, rafitilin, fructo oligosaccharides (NutraFlora), palatinose oligosaaccharides, guar gum hydrolysates (Sun Fiber) and dextrins are particularly suitable for the production of low-calorie chewing gums.

The selection of other flavors is practically unlimited and uncritical for the essence of the invention. Usually the total proportion of all flavors is 0.1 to 15 and preferably 0.2 to 5% by weight based on the chewing gum composition. Other suitable flavors include essential oils, synthetic flavors and the like, such as anise oil, star anise oil, caraway oil, eucalyptus oil, fennel oil, lemon oil, wintergreen oil, clove oil, and the like, which are also used in oral and dental care products.

Capsules

Capsules, which may contain the preparations according to the invention, are to be understood as spherical aggregates containing at least one solid or liquid core surrounded by at least one continuous shell. The active ingredients can be encapsulated by coating materials in the form of macrocapsules with diameters from about 0.5 to about 15 mm or micro-capsules with diameters from about 0.0001 to about 0.5 mm.

Coating Materials

Suitable coating materials include, for example, starches, including their degradation products and chemically or physically produced derivatives (in particular dextrins and malto-dextrins), gelatin, gum arabicum, agar-agar, ghatti gum, gellan gum, modified and non-modified celluloses, pullulan, curdlan, carrageenan, alginic acid, alginates, pectin, inulin, xanthan gum and mixtures of two or more of these substances.

The solid encapsulation material is preferably gelatin (in particular porcine, beef, poultry and/or fish gelatin), which preferably has a swelling factor of greater than or equal to 20, preferably greater than or equal to 24. Among these substances, gelatin is particularly preferred because it is readily available and can be obtained with various swell factors.

Maltodextrins (especially based on cereals, especially corn, wheat, tapioca or potatoes), which preferably have DE values in the range of 10 to 20, are also preferred. Further preferred are celluloses (e.g. cellulose ether), alginates (e.g. sodium alginate), carrageenan (e.g. beta-, jota-, lambda- and/or kappa-carrageenan), gum arabicum, curdlan and/or agar agar.

Alginate capsules as described in detail in the following publications are also preferred: EP 0389700 A1, U.S. Pat. No. 4,251,195, U.S. Pat. No. 6,214,376, WO 2003/055587 or WO 2004/050069 A1.

In another preferred embodiment, the shell of the capsules consists of coacervation products of cationic monomers or biopolymers (such as chitosan) and anionic monomers, such as (meth)acrylates or alginates.

Encapsulation Methods

The capsules are generally finely dispersed liquid or solid phases coated with film-forming polymers, whose preparation causes the polymers to deposit on the material to be encapsulated after emulsification and coacervation or interfacial polymerization. According to another method, molten waxes are taken up in a matrix (“microsponge”), which as micro-particles can additionally be coated with film-forming polymers. According to a third method, particles are coated alternately with polyelectrolytes of different charges (“layer-by-layer” method). The microscopically small capsules can be dried like powder. In addition to single-core microcapsules, multi-core aggregates, also known as microspheres, which contain two or more cores distributed in the continuous shell material are also known. Single or multi-core microcapsules can also be encapsulated by an additional second, third, etc., shell. The shell can be made of natural, semi-synthetic or synthetic materials. Natural shell materials include gum arabic, agar agar, agarose, maltodextrins, alginic acid and its salts, e.g. sodium or calcium alginate, fats and fatty acids, cetyl alcohol, collagen, chitosan, lecithin, gelatin, albumin, shellac, polysaccharides such as starch or dextran, polypeptides, protein hydrolysates, sucrose and waxes. Semi-synthetic coating materials include chemically modified celluloses, in particular cellulose esters and ethers, e.g. cellulose acetate, ethyl cellulose, hydroxypropyl cellulose, hydroxypropyl methyl cellulose and carboxymethyl cellulose, as well as starch derivatives, in particular starch ethers and esters. Synthetic coating materials include polymers such as polyacrylates, polyamides, polyvinyl alcohol or polyvinylpyrrolidone.

Examples of state of the art microcapsules include the following commercial products (the shell material is given in brackets): Hallcrest Microcapsules (gelatin, gum arabic), Coletica Thalaspheres (marine collagen), Lipotec Millicapsules (alginic acid, agar-agar), Induchem Unispheres (lactose, microcrystalline cellulose, hydroxypropyl methylcellulose); Unicerin C30 (lactose, microcrystalline cellulose, hydroxypropylmethylcellulose), Kobo Glycospheres (modified starch, fatty acid esters, phospholipids), Softspheres (modified agar agar) and Kuhs Probiol Nanospheres (phospholipids) as well as Primaspheres and Primasponges (chitosan, alginates) and Primasys (phospholipids).

Chitosan microcapsules and methods for their production are well known from the state of the art [WO 01/01926, WO 01/01927, WO 01/01928, WO 01/01929]. Microcapsules with mean diameters in the range of 0.0001 to 5, preferably 0.001 to 0.5 and in particular 0.005 to 0.1 mm, consisting of a shell membrane and a matrix containing the active ingredients, can be obtained by, for example

(a) preparing a matrix from gel formers, cationic polymers and active ingredients,
(b) optionally, dispersing the matrix in an oil phase,
(c) treating the dispersed matrix with aqueous solutions of anionic polymers, and optionally removing the oil phase.

Steps (a) and (c) are interchangeable in so far as anionic polymers can be used instead of cationic polymers in step (a) and vice versa.

The capsules can also be produced by alternately coating the active ingredient with layers of differently charged polyelectrolytes (layer-by-layer technology). In this context, reference is made to European Patent EP 1064088 B1 (Max Planck Society).

Medicaments

Another object of the present invention concerns preparations comprising

(a) omeprazole or pantoprazole
(b) eriodyctiol and/or homoeriodyctiol
as a medicament, especially for use as a medicament for treating inflammatory conditions of the stomach and intestines as well as reflux esophagitis. In particular, these products are intended for oral consumption. These products include tablets, capsules, chewable tablets, solutions, emulsions, suspensions, powders and granulates for direct ingestion, powders for dissolving in water, chewy sweets, lozenges, gum sweets, chewing gums, fruit gums and the like.

### INDUSTRIAL APPLICABILITY

Two other objects of invention concern, firstly, a method for masking bitter taste of pharmaceutical preparations containing omeprazole, comprising the following steps:

(i) providing a carrier material for the preparation of a pharmaceutical preparation;
(ii) adding to the material of step (i) an amount of omeprazole or pantoprazole;
(iii) adding to the mixture of step (ii) an amount of eriodyctiol and/or homoeriodyctiol; and
(iv) processing the mixture of step (iii) into a pharmaceutical end product, and secondly, the use of eriodyctiol and/or homoeriodyctiol to mask or inhibit bitter taste of omeprazole or pantoprazole.

The pharmaceutical end product may be a tablet, lollipop, chewing candy, chewing gum, fruit gum or capsule.

Insofar the above-mentioned preferred embodiments refer to mixtures of substances and amounts, these also apply for the claimed method respectively for the claimed use, so that a repetition is unnecessary.

### EXAMPLES 1 TO 3, COMPARATIVE EXAMPLES V1 and V2

To test the bitter masking effect, aqueous solutions, each containing 500 ppm omeprazole

(V1) alone or together
(1) with 100 ppm eriodyctiol (ED) or
(2) with 100 ppm homoeriodyctiol sodium salt (HE) or
(3) with 100 ppm eriodyctiol+100 ppm homoeriodyctiol sodium salt or
(V4) with 100 ppm 4′-fluoro-6-methoxyflavanone as a reference substance that has no masking effect, were tasted. Solutions 2-5 were each tasted by an experienced panel of 20-30 testers in a randomized order against solution 1 in duo comparison tests and the bitterness of each sample was estimated on a scale of 0 to 100. The mean values of the evaluations are summarized in Table 1.

TABLE 1
Evaluation of bitterness omeprazole

| | | | Reduction compared |
|---|---|---|---|
| Example | | Bitterness | to control example |
| V1 | Omeprazole | 80 | |
| 1 | Omeprazole + ED | 59 | −26 |
| 2 | Omeprazole + HE | 71 | −11 |
| 3 | Omeprazole + ED + HE | 44 | −45 |
| V2 | Omeprazole + 4′-fluoro-6- | 86 | +8 |
| | methoxyflavanone | | |

As can be seen, both ED and HE act on their own as masking agents for the bitterness of omeprazole. Surprisingly, the combination of the substances is with −45% clearly more effective than an additive masking of −37% to be expected. The structurally related reference compound in example V2 is not effective, but in most cases even increases the bitterness of omeprazole.

### APPLICATION EXAMPLES

### Application Example 1

Syrup with pantoprazole and ED/HED (amounts in % by weight)

| Ingredients | A | B |
|---|---|---|
| Glycerol | 10.0 | 10.0 |
| Sugar syrup | 45.0 | 45.0 |
| Pantoprazole 8% in water | 10.0 | 10.0 |
| Aspartame 5% in water | 5.0 | 5.0 |
| Symrise flavor | 0.4 | — |
| Symrise flavor with 1.25% ED | — | 0.4 |
| and 1.25% HED | | |
| water | Ad 100 | Ad 100 |

Preparation of a syrup with 40 mg pantoprazole/dose according to the above recipe. The preparations are thoroughly mixed until all components are dissolved. Compared to preparation A, preparation B was considerably less bitter.

### Application Example 2

Powder preparation with omeprazole and ED/HED (amounts in % by weight

| Ingredients | A | B |
|---|---|---|
| Omeprazole | 0.4 | 0.4 |
| Citric Acid Anhydrate | 3.0 | 3.0 |
| Aspartame | 2.5 | 2.5 |
| Ascorbic acid | 1.0 | 1.0 |
| Symrise flavor | 0.8 | — |
| Symrise flavor with 0.625% ED | — | 0.8 |
| and 0.625% HED | | |
| Saccharose | Ad 100 | Ad 100% |

Preparation of a powder mixture (5 g) with 20 mg omeprazole/dose according to the above recipe. The two preparations are mixed dry and then sieved. Compared to preparation A, preparation B was considerably less bitter.

### Application Example 3

Chewable tablet with omeprazole and ED/HED (amounts in % by weight)

| Ingredients | A | B |
|---|---|---|
| Calcium carbonate | 25.0 | 25.0 |
| Magnesium stearate | 0.5 | 0.5 |
| Omeprazole | 0.5 | 0.5 |
| Citric acid | 0.75 | 0.75 |
| Aspartame | 0.075 | 0.075 |
| Symrise flavor | 0.8 | — |
| Symrise flavor with 0.625% ED | — | 0.8 |
| and 0.625% HED | | |
| Dextrose | Ad 100 | Ad 100 |

Production of chewable tablets (2 g) with 10 mg omeprazole/dose according to the above recipe. All components are mixed and pressed into tablets after 1-2 h. Compared to preparation A, preparation B was considerably less bitter.

### Application Example 4

Effervescent tablet with Pantoprazole and ED/HED (amounts in % by weight)

| Ingredients | A | B |
|---|---|---|
| Omeprazole | 2.0 | 2.0 |
| Sorbitol | 8.4 | 8.4 |
| Sodium cyclamate | 1.5 | 1.5 |
| Saccharin | 0.25 | 0.25 |
| Propylene glycol | 0.625 | 0.625 |
| Symrise flavor | 0.8 | — |
| Symrise flavor with 0.625% ED | — | 0.8 |
| and 0.625% HED | | |

Preparation of an effervescent tablet (2 g) with 40 mg pantoprazole/dose according to the recipe above. All components are thoroughly mixed and then filled to 100% with a prepared mixture of sodium hydrogen carbonate and citric acid (in a weight ratio of 1:1.36).

After 1-2 h, it is sieved and then pressed into tablets. Compared to preparation A, preparation B was considerably less bitter.

1. A preparation comprising
(a) omeprazole and/or pantoprazole; and
(b) eriodyctiol and/or homoeriodyctiol.

2. The preparation according to claim 1 comprising components (a) and (b) in a weight ratio of about 4:1 to 400:1.

3. The preparation according to claim 1 comprising omeprazole in an amount of about 2,000 to about 20,000 ppm.

4. The preparation according claim 1 comprising eriodyctiol and/or homoeriodyctiol in an amount of about 50 to about 500 ppm.

5. The preparation according to claim 4 comprising eriodyctiol and homoeriodyctiol in an amount of about 25 to about 250 ppm.

6. The preparation according to claim 1 in solid form.

7. The preparation according to claim 6 that is a tablet or chewing gum.

8. The preparation according to claim 7, further comprising a carrier substance, a disintegrant, a colorant, and/or a flavoring substance.

9. The preparation according to claim 6 that is a capsule.

10. The preparation according to claim 6 comprising an acid-resistant coating or shell.

11. The preparation according to claim 1 that is a medicament.

12. (canceled)

13. (canceled)

14. The preparation according to claim 1 in the form of a tablet, a capsule, a chewable tablet, a solution, an emulsion, a suspension, a powder for direct ingestion, a soft candy, a lozenge, a gumdrop, a chewing gum, or a fruit gum.

15. A method for masking bitterness of a pharmaceutical preparation comprising omeprazole or pantoprazole, the method comprising:
(i) providing a carrier material for the preparation of a pharmaceutical preparation;
(ii) adding an amount of omeprazole or pantoprazole to the material of step (i);
(iii) adding an amount of eriodyctiol and/or homoeriodyctiol to the mixture of step (ii); and
(iv) processing the mixture of step (iii) into a pharmaceutical end product.

16. (canceled)

17. A method for treating an inflammatory condition of the stomach or intestines, or for treating reflux esophagitis, comprising administering a preparation according to claim 1 to a patient.

18. The method of claim 17, wherein the preparation is orally administered to the patient.

19. A method for masking or inhibiting bitterness of omeprazole or pantoprazole comprising combining eriodyctiol and/or homoeriodyctiol with the omeprazole or the pantoprazole.
